(19) United States
(12) Patent Application Publication
(10) Pub. No.: US 2023/0112567 A1
(43) Pub. Date: Apr. 13, 2023

(54) COMPOSITIONS FOR PREVENTION OF SIDE EFFECTS RELATED TO ALCOHOL CONSUMPTION

(71) Applicant: BuzzKill Beverages, LLC, Irmo, SC (US)

(72) Inventors: Terry Griffin, Middletown, OH (US); Herb Miller, Sarasota, FL (US)

(21) Appl. No.: 17/961,690

(22) Filed: Oct. 7, 2022

Related U.S. Application Data
(60) Provisional application No. 63/254,232, filed on Oct. 11, 2021.

(51) Int. Cl. A61K 31/455; A61P 25/32; A61K 9/00; A61K 31/7004; A61K 31/375; A61K 31/352; A61K 33/06; A61K 31/191; A61K 33/08; A61K 31/194; A61K 31/198; A61K 36/738; A61K 36/185; A61K 31/4415; A61K 31/675; A61K 31/385; A61K 31/525; A61K 36/28; A61K 36/67; A61K 36/899; A61K 38/16; A23L 33/125; A23L 33/15; A23L 33/16; A23L 33/105; A23L 33/175; A23L 33/21

(52) U.S. Cl. CPC A61K 31/455; A61P 25/32; A61K 9/0095; A61K 31/7004; A61K 31/375; A61K 31/352; A61K 33/06; A61K 31/191; A61K 33/08; A61K 31/194; A61K 31/198; A61K 36/738; A61K 36/185; A61K 31/4415; A61K 31/675; A61K 31/385; A61K 31/525; A61K 36/28; A61K 36/67; A61K 36/899; A61K 38/16; A23L 33/125; A23L 33/15; A23L 33/16; A23L 33/105; A23L 33/175; A23L 33/21; A23V 2002/00

(57) ABSTRACT

The present composition provides for mitigating adverse effects of alcohol consumption. The composition includes a fructose source comprising between 5% and 75% of a total weight of the composition, a niacinamide source comprising between 0.1% and 1% of the total weight, and a glycoprotein source comprising between 0.1% and 2% of the total weight, wherein the total weight is a weight prior to inclusion with a hydrating source. The composition can be disposed in a powder or dissolved form, as part of a concentrate, and/or part of a beverage. The composition can include mixability for diluting or mixing with the liquid or beverage for user consumption and absorprtion.

### BACKGROUND OF THE INVENTION

### Related Applications

The present application relates to and claims priority to U.S. Provisional Patent Application Ser. No. 63/254,232 filed Oct. 11, 2021, the entirety of which is hereby incorporated by reference.

### Field of the Invention

The present invention relates to a composition of matter for treatment and/or prevention of side effects related to alcohol consumption. More specifically, the use of compositions for treating, preventing, and/or alleviating hangover or symptoms thereof such as fatigue, weakness, excessive thirst, dry mouth, headache, muscle aches, nausea, vomiting, light sensitivity, sound sensitivity, dizziness, vertigo, shakiness, decreased ability to concentrate, mood disturbances, depression, anxiety, irritability, increased blood pressure, sweating, and/or rapid heartbeat.

### Description of Related Art

Each year, approximately 446 billion liters of alcoholic beverages are consumed worldwide. Beer is the most widely consumed alcoholic beverage worldwide (The Alcohol Industry in Data, Alcohol.org, 2021), and the third most popular beverage overall behind only water and tea. In 2018, beer saw a 5% increase in sales, with a record of over 7,000 breweries operating in the United States. Sales of distilled spirits, including whiskey, gin, rum, brandy, vodka, and tequila, have also experienced a steady growth rate, making up over a third of total alcohol sales.

After an alcoholic beverage is ingested, ethanol is rapidly absorbed into the blood (with approximately 20% absorbed by the stomach and 80% by the small intestine) and effects can be felt within 5 to 10 minutes. Ethanol levels typically peak in the blood approximately 60-90 minutes after ingestion. The liver, which is responsible for removing ethanol from the body, is capable of metabolizing about 1 standard drink per hour for men. The majority of ingested ethanol is metabolized in the liver by alcohol dehydrogenase (ADH). ADH is an enzyme that oxidizes ethanol into acetaldehyde, a known carcinogen. Acetaldehyde is further oxidized into acetate by aldehyde dehydrogenase (ALDH), before being decomposed into carbon dioxide and water. A small amount of ethanol is metabolized outside the liver, such as in the pancreas, brain, and gastrointestinal tract. When large amounts of alcohol are consumed, the enzyme cytochrome P450 2E1 (CYP2E1) also facilitates the transformation of ethanol into acetaldehyde. This process has been shown to produce reactive oxygen species (ROS), which are capable of damaging proteins and DNA (Alcohol Alert, U.S. Department of Health & Human Services, No. 72, April 2007).

Due to the effects of ethanol and its metabolites, it is beneficial to increase the speed at which the body removes ethanol after consumption of alcoholic beverages. Many factors can influence how fast ethanol is metabolized and how quickly those metabolites cleared from the body, such as gender, age, body mass, liver size, genetic differences in the ADH and/or ALDH enzymes (for example, ALDH2 deficiency commonly found in individuals of Asian descent), and other drug/medication use. In addition to increasing the rate at which ethanol is broken down by the liver, there is an interest in reducing an unwanted side effect of alcohol consumption, often referred to as hangover. Hangover symptoms include but are not limited to fatigue, weakness, excessive thirst, dry mouth, headache, muscle aches, nausea, vomiting, light sensitivity, sound sensitivity, dizziness, vertigo, shakiness, decreased ability to concentrate, mood disturbances, depression, anxiety, irritability, increased blood pressure, sweating, and/or rapid heartbeat. Hangovers are thought to have a variety of causes including but not limited to dehydration, sleep disruptions, inflammation, and/or acetaldehyde exposure (“Hangovers”, NIH: NIAA, March 2021).

Many different products are used by consumers to combat the side effects of alcohol consumption, but these products mostly treat the symptoms without preventing hangover or offering protection to the liver. For example, sports drinks and/or electrolyte solutions (such as Pedialyte) are often used to treat dehydration and electrolyte imbalance. While these products are useful in rehydrating the body and/or balancing electrolytes lost after consuming alcohol, dehydration is only one hangover contributor.

DRINKADE™, a product marketed as a hangover remedy, contains a proprietary blend comprising, among other ingredients, caffeine and aloe vera. These components have mixed results in improvement of hangover symptoms and potentially harm liver function. Another hangover treatment product, Blowfish™, is an alka-seltzer like tablet that seeks to treat hangover symptoms with active ingredients which include aspirin, caffeine, and an antacid. While each of these ingredients may relieve some hangover symptoms after they appear, the product does not offer preventative protection against hangover by enhancing metabolism of ethanol or preventing acetaldehyde build up in the liver.

A variety of patents and patent publications present compositions and beverages intended to mitigate the side effects of alcohol consumption. Some offer hepatoprotective properties, such as the herbal beverage found in KR0178696, which comprises the hepatoprotectant compound, diphenyl dimethyl bicarboxylate (DDB), the herbal drink of KR2001-0050333 which comprises a hepatoprotective extract from the Alder tree, and the soybean fermentation extract of KR2002-0021980. In another strategy for liver protection, the composition of KR2002-0004193 aims to prevent absorption of alcohol in the stomach. The composition of KR2004-0052930, which comprises artichoke extract powder, dandelion extract, and milk thistle extract, aims to reduce hangover symptoms by protecting the liver and offering a headache-reducing effect.

Other strategies toward preventing hangover are found in U.S. Pat. No. 7,544,356, which seeks to prevent hangover by improving liver function and reducing blood alcohol levels with a fermented composition with a variety of vitamins, extracts, and bacteria.

Still, a need exists for a composition comprising ingredients capable of preventing and/or treating the effects of alcohol consumption by accelerating the metabolism of alcohol, increasing the ability of the liver to remove acetaldehyde, providing protection to the liver and pancreas, and replenishing the body of vital nutrients.

### SUMMARY OF THE INVENTION

The present invention includes compositions, beverage concentrates, and/or beverages comprising ingredients capable of treating, alleviating, or preventing one or more side effects of alcohol consumption.

Embodiments include embodiment 1, which is a composition comprising one or more of fructose, niacinamide, and/or one or more source of glycoprotein, the composition consisting of a powder, a beverage concentrate, or a beverage when added with a hydrating source.

Embodiment 2 is the composition, beverage concentrate, or beverage of embodiment 1, wherein the source of glycoprotein is egg whites and/or eleuthero root extract.

Embodiment 3 is the composition of embodiments 1 or 2, further comprising feverfew leaf powder, rosemary, and/or magnesium.

Embodiment 4 is the composition of any embodiments 1-3, further comprising one or more of vitamin B1, vitamin C, Dihydromyricetin (DHM), taurine, stinging nettle, eleuthero extract, and/or glutathione.

Embodiment 5 is the composition of any embodiments 1-4, further comprising inulin.

Embodiment 6 is the composition of any embodiments 1-5, further comprising milk thistle extract.

Embodiment 7 is the composition of any embodiments 1-6, further comprising citrus bioflavonoid powder.

Embodiment is the composition of any embodiments 1-7, further comprising alpha-tocopheryl acetate.

Embodiment 9 is the composition of any embodiments 1-8, further comprising riboflavin.

Embodiment 10 is the composition of any embodiments 1-9, further comprising pyridoxine hydrochloride.

Embodiment 11 is the composition of any embodiments 1-10, further comprising alpha-lipoic acid.

Embodiment 12 is the composition of any embodiments 1-11, further comprising pyridoxine-5-phosphate.

Embodiment 13 is the composition of any embodiments 1-12, further comprising black pepper.

Embodiment 14 is the composition of any embodiments 1-13, further comprising citric acid.

Embodiment 15 is the composition of any Embodiments 1-14, further comprising ascorbic acid.

Embodiment 16 is the composition of any of embodiments 1-12, further comprising tricalcium phosphate.

Embodiment 17 is the composition of any embodiments 1-16, further comprising malic acid.

Embodiment 18 is the composition of any embodiments 1-17, further comprising one or more non-nutritive sweetener.

Embodiment 19 is the composition of any embodiments 1-18, further comprising one or more of sucralose, monk fruit, stevia, and/or aspartame.

Embodiment 20 is the composition of any embodiments 1-19, further comprising chromium picolinate.

Embodiment 21 is the composition of any of embodiments 1-20, further comprising stabilized rice bran.

Embodiment 22 is the composition any of embodiments 1-21, further comprising L-selenomethionine.

Embodiment 23 is a method of preventing one or more side effect of alcohol consumption comprising administering a composition, beverage concentrate, or beverage of any of Embodiments 1-22.

Embodiment 24 is a method of treating one or more side effect of alcohol consumption comprising administering a composition, beverage concentrate, or beverage of any of embodiments 1-22.

Embodiment 25 is the method of embodiment 23 or 24, wherein the one or more side effect of alcohol consumption is fatigue, weakness, excessive thirst, dry mouth, headache, muscle aches, nausea, vomiting, light sensitivity, sound sensitivity, dizziness, vertigo, shakiness, decreased ability to concentrate, mood disturbances, depression, anxiety, irritability, increased blood pressure, sweating, and/or rapid heartbeat.

Embodiment 26 is the method of any of embodiments 23-25, wherein the composition, beverage concentrate, or beverage is capable of increasing the rate at which the liver metabolizes ethanol and/or its metabolites.

Embodiment 27 is the method of any of embodiments 23-26, wherein the composition, beverage concentrate, or beverage is capable of protecting the liver from excessive build-up of acetaldehyde.

Embodiment 28 is the method of any of embodiments 23-27, wherein the composition, beverage concentrate, or beverage is capable of replenishing lost vitamins/minerals.

Embodiment 29 is the method of any of embodiments 23-28, wherein the composition, beverage concentrate, or beverage is capable of relieving pain of a subject.

Embodiment 30 is the method of any of embodiments 24-29, wherein the composition, beverage concentrate, or beverage is capable of increasing the rate at which ethanol is metabolized by up to 50%, such as up to 5%, up to 10%, up to 15%, up to 20%, up to 25%, up to 30% or up to 40%.

### DETAILED DESCRIPTION OF VARIOUS EMBODIMENTS OF THE INVENTION

The present invention aims to prevent, alleviate, and/or treat side effects of alcohol consumption, or hangover symptoms, in one or more of the following ways: 1) increasing the rate at which the liver metabolizes ethanol and/or its metabolites, 2) protecting the liver from excessive build-up of acetaldehyde, 3) replenishing lost vitamins/minerals, and/or 4) relieving pain.

In embodiments, the present invention comprises compositions, beverage concentrates, and beverages comprising ingredients capable of accelerating the metabolism of alcohol following the ingestion of an alcoholic beverage, increasing the ability of the liver to remove acetaldehyde in order to reduce the hangover effects common to alcohol consumption, and/or providing protection to the liver and pancreas.

In embodiments, the invention is administered to prevent, treat, alleviate, or reduce one or more of fatigue, weakness, excessive thirst, dry mouth, headache, muscle aches, nausea, vomiting, light sensitivity, sound sensitivity, dizziness, vertigo, shakiness, decreased ability to concentrate, mood disturbances, depression, anxiety, irritability, increased blood pressure, sweating, and/or rapid heartbeat associated with alcohol consumption.

In embodiments, the composition, beverage concentrate, or beverage comprises one or more component capable of improving liver function, such as by increasing the rate at which ADH and/or ALDH metabolize ethanol and/or acetaldehyde. Non-limiting examples of components that may improve liver function include one or more of fructose (or derivatives thereof), niacinamide (nicotinamide), glycoproteins (or sources of glycoproteins, for example, egg whites, which contain ovomucin), and/or eleuthero extract. In embodiments, the fructose is crystalline fructose.

In embodiments, the composition, beverage concentrate, or beverage comprises one or more ingredient capable of protecting the liver from acetaldehyde. Non-limiting examples of ingredients that may protect the liver from a build of up acetaldehyde include vitamin B1 (thiamine), vitamin C, Dihydromyricetin (DHM), taurine, stinging nettle, eleuthero, and/or glutathione.

In embodiments, the composition, beverage concentrate, or beverage comprises one or more component capable of providing pain relief. Non-limiting examples of components that may act as pain relievers include feverfew extract, rosemary, and/or magnesium.

In the following paragraphs, the weight percentages are given for a variety of ingredients that may be present in the composition. The weight percent given describes the amount of the ingredient that may be present in the composition, beverage concentrate, or beverage, excluding added water. For example, a 24 g dose of the composition with 50 weight percent of an ingredient A comprises 12 g of ingredient A. In another example, a 12 fluid ounce beverage comprises, for example, 24 g of ingredients which are diluted with water to a final volume of 12 fluid ounces. If the beverage has 50 weight percent of an ingredient A, the beverage comprises 12 g of ingredient A. The term “about” used herein in the context of quantitative measurements means the indicated amount ±10%. For example, with a ±10% range, “about 2 weight percent” can mean 1.8-2.2 weight percent.

In embodiments, the composition, beverage concentrate, or beverage comprises fructose and/or crystalline fructose in an amount of about 5 to about 75 weight percent, such as about 10, 15, 20, 25, 30, 35, 40, 45, 50, 55, 60, 65, or 70 weight percent.

In embodiments, the composition, beverage concentrate, or beverage comprises one or more sources of fiber, such as inulin or sources of inulin (for example, inulin from asparagus, bananas, burdock, chicory, dandelion root, garlic, Jerusalem artichokes, leeks, or onions), in an amount of about 1 to about 30 weight percent, such as about 2, 3, 4, 5, 6, 7, 8, 9, 10, 11, 12, 13, 14, 15, 16, 17, 18, 19, 20, 25, or 27 weight percent.

In embodiments, the composition, beverage concentrate, or beverage comprises one or more flavoring agents (such as natural flavors) and/or preserving agents, such as citric acid, each in an amount of about 1 to about 20 weight percent, such as about 2, 3, 4, 5, 6, 7, 8, 9, 10, 11, 12, 13, 14, 15, 16, 17, 18, or 19 weight percent.

In embodiments, the composition, beverage concentrate, or beverage comprises Dihydromyricetin (DHM) in an amount of up to about 30 weight percent, such as about 0.5, 0.75, 1, 1.25, 1.5, 1.75, 1.9, 2, 2.1, 2.25, 2.5, 3, 3.5, 4, 5, 6, 7, 8, 9, etc., weight percent.

In embodiments, the composition, beverage concentrate, or beverage comprises ascorbic acid (vitamin C) in an amount of up to about 5 weight percent, such as about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.25, 2.5, 3, 3.5, 4, or 4.5 weight percent.

In embodiments, the composition, beverage concentrate, or beverage comprises one or more anti-caking agent, such as tricalcium phosphate, in an amount of up to about 5 weight percent, such as about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.25, 2.5, 3, 3.5, 4, or 4.5 weight percent.

In embodiments, the composition, beverage concentrate, or beverage comprises milk thistle extract (silymarin) in an amount of up to about 5 weight percent, such as about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.25, 2.5, 3, 3.5, 4, or 4.5 weight percent.

In embodiments, the composition, beverage concentrate, or beverage comprises one or more of eleuthero root extract, feverfew leaf powder, malic acid, and/or taurine each in an amount of up to about 5 weight percent, such as about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.25, 2.5, 3, 3.5, 4, or 4.5 weight percent.

In embodiments, the composition, beverage concentrate, or beverage comprises one or more sources of minerals, such as magnesium (as magnesium gluconate, magnesium oxide, magnesium hydroxide, magnesium chloride, magnesium citrate salts, or magnesium aspartate), in an amount of up to about 2 weight percent, such as up to about 0.1, 0.2, 0.3, 0.4, 0.5, 0.6, 0.7, 0.8, 0.9, 1, 1.1, 1.2, 1.3, 1.4, 1.5, 1.6, 1.7, 1.8, or 1.9 weight percent.

In embodiments, the composition, beverage concentrate, or beverage comprises rosemary herb powder in an amount of up to about 2 weight percent, such as up to about 0.1, 0.2, 0.3, 0.4, 0.5, 0.6, 0.7, 0.8, 0.9, 1, 1.1, 1.2, 1.3, 1.4, 1.5, 1.6, 1.7, 1.8, or 1.9 weight percent.

In embodiments, the composition comprises one or more glycoprotein and/or source of glycoprotein in an amount of up to about 2 weight percent, such as up to about 0.1, 0.2, 0.3, 0.4, 0.5, 0.6, 0.7, 0.8, 0.9, 1, 1.1, 1.2, 1.3, 1.4, 1.5, 1.6, 1.7, 1.8, or 1.9 weight percent. Non-limiting examples of glycoproteins include egg whites (ovomucin), miraculin, pectin, brans, arabinogalactan, seaweeds, green ocean algae, etc.

In embodiments, the composition, beverage concentrate, or beverage comprises one or more of thiamin (vitamin B1), thiamin hydrochloride, niacinamide (vitamin B3), tocopheryl acetate (vitamin E), and/or L-glutathione each in an amount of up to about 1 weight percent, such as up to about 0.05, 0.1, 0.15, 0.2, 0.25, 0.3, 0.35, 0.4, 0.45, 0.5, 0.6, 0.7, 0.8, or 0.9 weight percent.

In embodiments, the composition, beverage concentrate, or beverage comprises one or more bioflavonoid or sources of bioflavonoids (non-limiting examples of which include citrus bioflavonoid complex, rose hips, or black currants) in an amount of up to about 1 weight percent, such as up to about 0.05, 0.1, 0.15, 0.2, 0.25, 0.3, 0.35, 0.4, 0.45, 0.5, 0.6, 0.7, 0.8, or 0.9 weight percent.

In embodiments, the composition, beverage concentrate, or beverage comprises stabilized rice bran in an amount of up to about 0.5 weight percent, such as up to about 0.01, 0.02, 0.03, 0.04, 0.05, 0.06, 0.07, 0.08, 0.09, 0.1, 0.15, 0.2, 0.25, 0.3, 0.35, 0.4, or 0.45 weight percent.

In embodiments, the composition, beverage concentrate, or beverage comprises one or more of vitamin B6 (such as in the form of pyridoxine hydrochloride and/or pyridoxine-5-phosphate) and/or alpha-lipoic acid each in an amount of up to about 0.5 weight percent, such as up to about 0.01, 0.0125, 0.015, 0.02, 0.025, 0.03, 0.035, 0.04, 0.045, 0.05, 0.06, 0.07, 0.08, 0.09, 0.1, 0.15, 0.2, 0.25, 0.3, 0.35, 0.4, or 0.45 weight percent.

In embodiments, the composition, beverage concentrate, or beverage comprises one or more of black pepper and/or riboflavin (vitamin B2) each in an amount of up to about 0.1 weight percent, such as up to about 0.0024, 0.003, 0.004, 0.005, 0.0075, 0.01, 0.025, 0.03, 0.04, 0.05, 0.06, 0.075, 0.08, 0.09, or 0.095 weight percent.

In embodiments, the composition, beverage concentrate, or beverage comprises one or more of chromium picolinate and/or L-selenomethionine each in an amount of up to about 0.05 weight percent, such as up to about 0.0001, 0.00025, 0.0003, 0.0005, 0.00075, 0.0009, 0.001, 0.0025, 0.005, 0.0075, 0.01, 0.02, 0.03, or 0.04 weight percent.

In embodiments, the composition, beverage concentrate, or beverage comprises one or more natural or artificial sweetener such as aspartame, saccharin, sucralose, sugar alcohols (erythritol, xylitol, or sorbitol), monk fruit extract, and/or stevia. In embodiments, the one or more artificial sweetener is present in the composition in an amount of up to about 5 weight percent, such as about 0.01, 0.05, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.25, 2.5, 3, 3.5, 4, or 4.5 weight percent.

In embodiments, the composition, beverage concentrate, or beverage comprises one or more fruit and/or vegetable extract, powder, concentrate, puree, juice, juice powder, or juice blend. Non-limiting examples of said fruits and vegetables include acai, apple, apricot, avocado, banana, bean, beet, blueberry, cabbage, cantaloupe, carrot, celery, cherry, cucumber, date, dragon fruit, fig, grapefruit, grape, guava, honeydew, kiwi, lemon, lime, lychee, mandarin, mango, nectarine, orange, papaya, passion fruit, peach, pear, pineapple, plum, pomegranate, prune, pumpkin, radish, raspberry, rhubarb, rutabaga, seaweed, squash, strawberry tangelo, tangerine, tomato, turnip, watermelon, and/or zucchini, or combinations thereof. In embodiments, the one or more fruit and/or vegetable extract, powder, concentrate, puree, juice, juice powder, or juice blend is present in the composition in an amount of up to about 25 weight percent, such as up to about 0.1, 0.5, 1, 2, 3, 4, 5, 6, 7, 8, 9, 10, 12, 15, 18, 20, 22, or 24 weight percent. In some embodiments, the composition, beverage concentrate, or beverage contains 1, 2, 3, 4, 5, or more vegetable extract, powder, concentrate, puree, juice, juice powder, or juice blend.

In embodiments, the composition, beverage concentrate, or beverage comprises one or more natural or artificial food coloring, such as annatto extract, beet juice, beta carotene, black or purple carrot, blue spirulina extract, calcium carbonate, caramel color, elderberry, hibiscus fruit, paprika, purple carmine, purple sweet potato, red cabbage, red carmine, red radish, riboflavin, titanium dioxide, turmeric (curcumin) or combinations thereof. In embodiments, the natural or artificial food coloring is present in the composition, beverage concentrate, or beverage in an amount of up to about 5 weight percent, such as about 0.01, 0.05, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.25, 2.5, 3, 3.5, 4, or 4.5 weight percent.

In embodiments, the composition, beverage concentrate, or beverage is capable of decreasing the half-life of ethanol in the blood by about 5% to about 100%, such as about 10%, 20%, 30%, 40%, 50%, 60%, 70%, 80% or 90%.

In an embodiment of the invention, two or more of the components are present in the composition in synergistically effective amounts.

In embodiments, the composition can be formulated as a solid oral dosage formulation, such as in the form of tablets, caplets, pills, capsules, or the like. In embodiments, capsules may be formulated by mixing the composition with a pharmaceutical diluent and inserting the combination into a soft or hard capsule. In other embodiments, the composition can be formulated as chewable tablets or gummies (gelatin-based or non-gelatin based).

A hydrating source may be utilized for facilitating user consumption. Where the composition is a powder, solid oral dosage, soft or hard capsule, chewable tablet or gummy, a hydrating agent may be excluded from the composition assembly and weight percentages. Where the composition is a beverage concentrate, the hydrating source can be one or more liquids for mixing the composition into its concentrate form. Where the composition is a beverage, the hydrating source can be one or both liquids for converting the composition of pre-hydrating source weights into the beverage, for example a hydrating source can be water for mixing the composition and then a second liquid source can be the base beverage liquid, e.g. water, milk, tea, coffee, etc.

The composition can also be formulated as a beverage concentrate to be dissolved and/or diluted with a liquid prior to ingestion by a user. In embodiments, the beverage concentrate is a syrup or powder concentrate. For example, one dose of the powdered concentrate can be dissolved in water, or other liquid such as tea, coffee, milk, etc., to form a beverage. In a preferred embodiment, the beverage concentrate can be dissolved in about 8 oz. to 16 oz. of water or other liquid, such as 8 oz. to 9 oz., 9 oz. to 10 oz., 10 oz. to 11 oz., 11 oz. to 12 oz., 12 oz. to 13 oz., 13 oz. to 14 oz., 14 oz. to 15 oz., or 15 oz. to 16 oz.

In embodiments, the inventive composition is formulated as a beverage comprising water in an amount of up to about 99 weight percent, such as about 5, 10, 15, 20, 25, 30, 35, 40, 45, 50, 55, 60, 65, 70, 75, 80, 85, 86, 87, 88, 89, 90, 91, 92, 93, 94, 95, 96, 97, or 98 weight percent.

In any of the embodiments described, the pH of the beverage can be in the range of about 2 to about 9, such as about 2.5, 3, 3.5, 4, 4.5, 5, 5.5, 6, 6.5, 7, 7.5, 8, or 8.5.

In embodiments, the beverage comprises caffeine. In some embodiments, the beverage is carbonated.

In embodiments, the composition, beverage concentrate, or beverage is capable of reducing the blood alcohol concentration of a subject if administered before, during, or after the subject ingests alcohol.

In embodiments, the composition, beverage concentrate, or beverage is capable of providing protection to the liver and/or pancreas.

In embodiments, the composition, beverage concentrate, or beverage is capable of relieving or preventing headaches.

In embodiments, the composition, beverage concentrate, or beverage is capable of reducing the amount of alcohol absorbed by the stomach and/or gastrointestinal system.

The following examples are illustrative and should not be interpreted to limit the scope of the claimed subject matter.

### EXAMPLE 1

In an example protocol, a male subject, age 25, weighing 165 pounds is administered 6 ounces of hard liquor (40% alcohol by volume). An estimated maximum blood alcohol concentration (BAC) of the subject is approximately 0.120%. Using an estimated ethanol clearance rate of 0.015% per hour, two hours after ingesting the liquor the subject's estimated BAC would be approximately 0.090%. It would take the subject approximately 8 hours to return to a blood alcohol concentration of 0%.

An example protocol for how a hangover prevention composition could be used is described herein. The same male subject, prior to ingesting the 6 ounces of hard liquor, is given a 12 ounce serving of the inventive beverage, according to an embodiment of the invention. After 30 minutes, the subject ingests the 6 ounces of hard liquor. An estimated maximum blood alcohol concentration of the subject is up to 0.120%. Approximately two hours after ingesting the liquor, the subject's estimated BAC would be approximately 0.084%. The estimated ethanol clearance rate is up to 0.018% per hour. As such, administration of the inventive beverage would be expected to result in the subject's BAC returning to zero up to 20% more quickly than it would without the beverage. Administration of the inventive beverage would also be expected to result in a lower maximum blood alcohol concentration of a subject, if administered prior to alcohol ingestion.

The present invention has been described with reference to particular embodiments having various features. In light of the disclosure provided above, it will be apparent to those skilled in the art that various modifications and variations can be made in the practice of the present invention without departing from the scope or spirit of the invention. One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Any of the methods disclosed herein can be used with any of the compositions disclosed herein or with any other compositions. Likewise, any of the disclosed compositions can be used with any of the methods disclosed herein or with any other methods. Other embodiments of the invention will be apparent to those skilled in the art from consideration of the specification and practice of the invention.

It is noted in particular that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all of the references cited in this disclosure are each individually incorporated by reference herein in their entireties and as such are intended to provide an efficient way of supplementing the enabling disclosure of this invention as well as provide background detailing the level of ordinary skill in the art.

What is claimed is:

1. A composition designed for mitigating adverse effects of alcohol consumption, the composition comprising:
a fructose source comprising between 5% and 75% of a total weight of the composition;
a niacinamide source comprising between 0.1% and 1% of the total weight; and
a glycoprotein source comprising between 0.1% and 2% of the total weight;
wherein the total weight is a weight prior to inclusion with a hydrating source.

2. The composition of claim 1 further comprising:
an ascorbic acid source comprising between 0.5% and 5% of the total weight.

3. The composition of claim 1 further comprising:
a fiber source comprising between 1.0% and 30% of the total weight, wherein the weight does not exceed 100%.

4. The composition of claim 1 further comprising:
a flavoring agent comprising between 1% and 20% of the total weight.

5. The composition of claim 1 further comprising:
a dihydromyricetin (DHM) comprising between 0.1% and 30% of the total weight.

6. The composition of claim 1 further comprising:
at least one mineral source comprising 0.1% and 2% of the total weight;
wherein the at least one mineral source includes one or more of: magnesium, magnesium gluconate, magnesium oxide, magnesium hydroxide, magnesium chloride, magnesium citrate salts, and magnesium aspartate.

7. The composition of claim 1 further comprising:
at least one bioflavonoid source comprising up to 1% of the total weight;
wherein the bioflavonoid is one or more of: citrus bioflavonoid complex, rose hips, and black currants.

8. The composition of claim 1 further comprising:
stabilized rice bran source comprising up to 0.5% of the total weight.

9. The composition of claim 1 further comprising:
at least one vitamin B6 source comprising up to 0.5% of the total weight;
wherein the vitamin B6 source is one or more of: pyridoxine hydrochloride, pyrodixine-5-phosphate, and alpha-lipoic acid.

10. The composition of claim 1 further comprising:
black pepper comprising up to 0.1% of the total weight.

11. The composition of claim 1 further comprising:
a vitamin B2 source comprising up to 0.1% of the total weight.

12. The composition of claim 1 further comprising:
a chromium picolinate source comprising up to 0.05% of the total weight.

13. The composition of claim 1 further comprising:
an L-selenomethionine source comprising up to 0.05% of the total weight.

14. The composition of claim 1 further comprising:
a sweetener comprising up to 5% of the total weight;
wherein the sweetener includes one or more of: aspartame, saccharin, sucralose, sugar alcohols, monk fruit extract, and stevia.

15. The composition of claim 1 further comprising:
milk thistle extract comprising up to 5% of the total weight.

16. The composition of claim 1 further comprising:
an fruit/vegetable extract source comprising up to 25% of the total weight.

17. The composition of claim 1, wherein the hydrating source includes a liquid mixable with the fructose source, the niacinamide source, and the glycoprotein source such that the composition is formulated as a beverage concentrate to be dissolved in the hydrating source.

18. The composition of claim 17, wherein the beverage concentrate is formulated as at least one of: a syrup and a powder.

19. The composition of claim 1, wherein the hydrating source includes a liquid mixable with the fructose source, the niacinamide source, and the glycoprotein source such that the composition is formulated as a beverage concentrate to be diluted with a second liquid.

20. The composition of claim 19, wherein the liquid is water and the second liquid is at least one of: water, tea, coffee, fruit juice, and vegetable juice.
